(19) United States
(12) Patent Application Publication
(10) Pub. No.: US 2006/0003919 A1
(43) Pub. Date: Jan. 5, 2006

(54) Cosmetic/dermatological applications of LIF

(76) Inventors: Nicolas Fortunel, Breuillet (FR); Dominique Bernard, Paris (FR); Corinne Ferraris, Paris (FR); Marcelle Regnier, Paris (FR)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(21) Appl. No.: 11/137,344

(22) Filed: May 26, 2005

Related U.S. Application Data
(60) Provisional application No. 60/641,434, filed on Jan. 6, 2005.

(30) Foreign Application Priority Data
May 26, 2004 (FR) 04/51034

(51) Int. Cl. A61K 38/18; A61K 7/06; A61K 7/11

(52) U.S. Cl. 514/2; 424/70.14

(57) ABSTRACT

Leukemia inhibitory factor (“LIF”), an LIF analogue, an LIF mimetic, a product capable of stimulating the expression of endogenous LIF and mixtures thereof are useful for maintaining and/or stimulating the regenerative power of a population of human undifferentiated skin stem cells and/or epidermal progenitors, and are also useful for treating dry and/or chapped and/or aged skin and/or hair loss, as well as for treating skin burns and promoting skin regeneration and/or cicatrization.

### CROSS-REFERENCE TO PRIORITY/PROVISIONAL APPLICATIONS

This application claims priority under 35 U.S.C. § 119 of FR 04/51034, filed May 26, 2004, and of provisional application Ser. No. 60/641,434, filed Jan. 6, 2005, each hereby expressly incorporated by reference and each assigned to the assignee hereof. This application is also a continuation of said '434 provisional.

### CROSS-REFERENCE TO COMPANION APPLICATION

Our copending application Serial No. ______ [Attorney Docket No. 016800-737], filed concurrently herewith and also assigned to the assignee hereof.

### BACKGROUND OF THE INVENTION

### TECHNICAL FIELD OF THE INVENTION

The present invention relates to applications of LIF (leukemia inhibitory factor), of a LIF analogue or of a LIF mimetic, in cosmetics and in dermatology.

The present invention relates in particular to the formulation, into cosmetic compositions, of an effective amount of a compound selected from among LIF, a LIF analogue, a LIF mimetic and a product capable of stimulating the expression of endogenous LIF, and mixtures thereof, as an active agent suited to maintain and/or to stimulate the regenerative power of a population of human undifferentiated skin stem cells and/or epidermal progenitors.

According to the invention, the expression “regenerative power of a population of human undifferentiated skin stem cells and/or epidermal progenitors” means, in particular, (i) the capacity of said population of cells for self-renewal and/or their capacity to proliferate, and/or (ii) their capacity to regenerate a pluristratified epithelium, in particular a pluristratified epidermis and/or all or some of the skin appendages (sebaceous glands, hair follicle, nails, etc.).

According to the invention, the expression “population of human undifferentiated skin stem cells and/or epidermal progenitors” means a population of adult somatic stem cells naturally present in the basal layer of the epidermis, which are capable of self-renewal and/or of proliferating, and of generating a pluristratified epithelium, in particular a pluristratified epidermis, and/or all or some of the skin appendages (sebaceous glands, hair follicle, nail, etc.).

The expression “capacity of a cell for self-renewal” means a cell capable of dividing so as to give two daughter cells, at least one of which is identical to the mother cell. On the scale of a complex cell population, the notion of self-renewal implies the maintenance of a compartment of cells having phenotypic and functional characteristics that are constant in the course of the successive cell divisions. According to the invention, this will involve the maintenance of a compartment made up of undifferentiated skin stem cells and/or epidermal progenitors, in particular characterized by a strong proliferative potential and a capacity to generate a pluristratified epithelium, in particular a pluristratified epidermis.

The expression “capacity of a cell to proliferate” means a cell capable of multiplying to give two daughter cells, without there necessarily being transmission of the characteristics and of the potential of the mother cell to at least one of the two daughter cells. The proliferation, which may or may not be associated with the self-renewal phenomenon, is liable to result in the gradual decrease or the disappearance of the cellular compartment of interest within the cell population that multiplies. According to the invention, the cell proliferation can be accompanied by a gradual decrease in and/or by the disappearance of the compartment of undifferentiated skin stem cells and/or epidermal progenitors, in particular characterized by a strong proliferative potential and a capacity to generate a pluristratified epithelium, in particular a pluristratified epidermis.

This population comprises, in particular, undifferentiated skin stem cells and/or epidermal progenitors.

The stem cell compartment, located most upstream in the tissue hierarchy, is characterized by a strong potential for self-renewal (most substantial capacity for long-term multiplication), and also by a multi-potentiality (especially a capacity to produce a pluristratified epithelium, in particular keratinocytes and/or various cell types of the skin appendages, such as hair follicles, sebaceous glands, nails, etc.).

The epidermal progenitors, derived from said stem cells, exhibit a multiplication potential which can also be considerable, but generally less than that of the stem cells, and are characterized by a capacity to generate a pluristratified epithelium, in particular a pluristratified epidermis.

A quantitative and/or qualitative decrease in the pool of skin stem cells and/or of epidermal progenitors, and also an impairment of their cellular and molecular environment necessary for their stimulation, could be one of the possible causes of skin aging.

Hence the search for agents capable of maintaining and/or increasing this pool of skin stem cells and/or of epidermal progenitors, in order to promote epidermal renewal and prevent and/or combat cutaneous signs of aging.

### SUMMARY OF THE INVENTION AND DESCRIPTION OF BACKGROUND AND/OR RELATED AND/OR PRIOR ART

The present invention features the formulation of a compound selected from among LIF, a LIF analogue, a LIF mimetic and a product capable of stimulating the expression of endogenous LIF, and mixtures thereof, into compositions suited to promote skin regeneration and cicatrization, in particular for the treatment of burns.

This invention also features cosmetic or pharmaceutical compositions containing at least one compound selected from among LIF, a LIF analogue, a LIF mimetic and a product capable of stimulating the expression of endogenous LIF, and mixtures thereof, and to cosmetic processes (regime or regimen) for the treatment of dry and/or chapped and/or aged skin, or to cosmetic processes for the treatment of hair loss.

The skin constitutes a physical barrier between the organism and its environment. It consists of two tissues: the epidermis and the dermis.

The dermis gives the epidermis a solid support. It is also its nourishing factor. It consists mainly of fibroblasts and of an extracellular matrix that is itself composed in particular of collagen, of elastin, of fibronectin and of a substance known as ground substance, these components being essentially synthesized by the fibroblasts. Leukocytes, mast cells or tissue macrophages are also found therein. It also contains blood vessels and nerve fibers.

The epidermis is a desquamating pluristratified epithelium, 100 μm thick, on average, and is conventionally divided up into a basal layer containing skin stem cells, undifferentiated epidermal progenitors, and also cells involved in the maturation/differentiation process, a “spiny” layer consisting of several layers of polyhedral cells placed on the basal cells, a “granular” layer consisting of flattened cells containing cytoplasmic inclusions, keratohyalin granules, and, finally, an upper layer called horny layer (or stratum corneum), consisting of keratinocytes at the terminal stage of their differentiation, called corneocytes. The latter are mummified, anuclear keratinized cells that derive from keratinocytes. The stacking of the corneocytes constitutes the horny layer which performs, inter alia, the barrier function of the epidermis.

Epidermal differentiation follows a process of continuous and oriented maturation in which the basal keratinocytes transform while migrating so as to result in the formation of corneocytes, dead cells that are completely keratinized. This differentiation is the result of perfectly coordinated phenomena which will result in a constant thickness being maintained and will thus ensure the homeostasis of the epidermis. This means that there must be a precise regulation of the number of cells that enter into the differentiation process and of the number of cells that desquamate. In the course of the normal desquamation process, only the most superficial corneocytes detach from the surface of the epidermis.

Various physiological parameters are associated with skin “aging”. It is in particular known that, during chronobiological aging, the thickness of the epidermis decreases. The regenerative potential of the epidermis becomes less substantial since the cells of the basal layer comprising in particular the stem cells and the epidermal progenitors divide less actively, resulting in particular in a slowing down of and/or a decrease in epidermal renewal. It has also been described that, at the menopause, skin aging accelerates and the thickness of the skin decreases. Women complain that their skin tightens and becomes dry, or even that xerosis appears. The hormone deficiencies associated with the menopause are accompanied in particular by a decrease in metabolic activity, which could result in a decrease in the proliferation of keratinocytes.

Skin “aging” is also associated with environmental causes. Among the most common, it is in particular known that prolonged and/or repeated exposure to the sun results in quite similar results on the epidermis. This is photo-induced aging.

The cutaneous signs of aging result in particular in a thinning of the skin and/or a loss of firmness, of elasticity and/or of tonicity of the skin, and/or the formation of wrinkles and fine lines. The microrelief of the skin, consisting of microdepressions at the surface of the skin, is more accentuated and the appearance of the skin is less smooth. The wrinkles and fine lines appear in particular at the nasolabial fold, the crow's feet, the forehead, around the mouth and on the neck.

It is also known that, in the case of certain diseases such as psoriasis or ichthyosis, the skin exhibits alterations associated with an altered cell proliferation and/or differentiation.

The need for having agents capable of stimulating the multiplication of epidermal cells in order to facilitate the regeneration of the epidermis and to give the skin a young appearance is therefore apparent. One also appreciates a measure of the extent to which such active agents could be used in the case of skin cicatrization and regeneration, in particular for the treatment of burns.

Compounds capable of stimulating the proliferation of mature keratinocytes and thus promoting epidermal renewal are known from the prior art, such as for example retinoids, vitamin A derivatives, minerals and trace elements.

However, it has now unexpectedly been determined that it is also possible to maintain and/or increase the regenerative power of a population of undifferentiated skin stem cells and/or epidermal progenitors by administering leukemia inhibitory factor (LIF).

According to the invention, the expression “to maintain and/or increase the regenerative power of a population of undifferentiated skin stem cells and/or epidermal progenitors” means an agent capable in particular of:
(i) promoting the capacity of undifferentiated skin stem cells and/or epidermal progenitors for self-renewal and/or their capacity to proliferate;
(ii) and/or of maintaining and/or of increasing their capacity to generate a pluristratified epithelium, in particular a pluristratified epidermis, and/or to generate all or some of the skin appendages.

According to the invention, said expression also means an agent capable of:
(i) promoting the capacity of undifferentiated skin stem cells and/or epidermal progenitors for self-renewal and/or their capacity to proliferate;
(ii) and/or of maintaining and/or of increasing their ability to generate a pluristratified epithelium, in particular a pluristratified epidermis, and/or to generate all or some of the skin appendages, in response to an exogenous stimulus and/or in response to a signal derived from the physiological environment of the cells in vivo.

In fact, the action of LIF, of a LIF analogue or of a LIF mimetic may be direct, by stimulation of the regenerative potential of the skin stem cells and/or the epidermal progenitors. It may be also be indirect, in particular via factors and/or signals emitted by LIF-reactive cells, and capable of beneficially stimulating the neighboring cells. They are, for example, signals transmitted by direct contact between the cells, and/or paracrine regulations. The good health of the skin and also the regulation of its regenerative potential involve in particular this type of “dialogue” between the fibroblasts of the dermis and the epidermal progenitors and/or stem cells. Said cells will, consequently, be more receptive to exogenous stimuli and/or to a signal derived from the physiological environment of the cells in vivo.

Epidermal homeostasis results in particular from a finely regulated balance between mitogenic signals that promote cell division and anti-proliferative signals. These signals result in particular from the action of the factors naturally produced by the keratinocytes and/or by the other cell types present in their environment, and in particular secreted by the fibroblasts of the dermis.

As examples of “stimuli and/or signals known to be involved in epidermal homeostasis”, mention may in particular be made of:
growth factors, such as the mitogenic growth factors epidermal growth factor (EGF) and keratinocyte growth factor (KGF) (Cook et al., J. Cell Physiol., 146: 277-289, 1991; Andreadis et al., FASEB J., 15: 898-906, 2001; Gamady et al., J Cell Biochem., 89: 440-449, 2003), or else transforming growth factor-β1 (TGF-β31), a multifunctional growth factor identified in particular for its anti-proliferative effect on keratinocytes in vivo and in vitro (Glick et al., Proc. Natl. Acad. Sci. USA., 90: 6076-6080, 1993; Van Ruissen et al., J Cell Sci., 107: 2219-2228, 1994; Cui et al., Genes Dev., 9: 945-955, 1995). These factors are involved in the highly interactive and interlinked autocrine and paracrine regulatory loops, ensuring both the control of proliferation and that of differentiation (Reiss & Sartorelli, Cancer Res., 47: 6705-6709, 1987; Hertle et al., J. Invest. Dermatol, 104: 260-265, 1995; Edmonson et al., J. Cell Physiol., 179: 201-207, 1999; Yamasaki et al., J. Invest. Dermatol., 120: 1030-1037, 2003; Pasonen-Seppanen et al., J. Invest. Dermatol., 120: 1038-1044, 2003), and thus allowing precise regulation of the renewal and/or of the maturation of keratinocytes; and/or
of molecules that can complete, modulate and/or interfere with the action of growth factors, such as, for example: vitamin D and its derivatives for their ability to increase keratinocyte sensitivity to the mitogenic action of KGF (Gamady et al., J. Cell Biochem., 89: 440-449, 2003); retinoic acid for its ability to modulate keratinocyte proliferation and/or differentiation (Choi & Fuchs, Cell Regul, 1: 791-809, 1990; Gibbs et al., Arch. Dermatol. Res., 288: 729-738; 1996; Chapellier et al., EMBO J., 21: 3402-3413, 2002), in particular by means of a mechanism of sensitization of the cells to the action of EGF and of TGF-β (Tong et al., J. Invest. Dermatol., 94: 126-131, 1990).

The advantage of administering LIF, a LIF analogue, a LIF mimetic or a product capable of stimulating the expression of endogenous LIF, or mixtures thereof, for stimulating the regenerative power of said undifferentiated skin stem cells and/or epidermal progenitors capable of generating a pluristratified epidermis and/or all or some of the skin appendages, in “anti-aging” or “anti-hair loss” cosmetic compositions, and/or for preparing pharmaceutical compositions suited for skin regeneration and/or cicatrization, is therefore also apparent.

This novel strategy targets populations of cells located further upstream in the epidermal tissue hierarchy than any cell population made up of mature keratinocytes, differentiated keratinocytes or keratinocytes in the differentiation pathway. It therefore has the advantage of opening up the possibility of stimulating the renewal and/or the regeneration of all or some of the components of skin tissue, in particular of the epidermis, in a manner that is more effective and longer lasting than by targeting cell populations that are more mature, differentiated, or in the differentiation pathway.

LIF is described in U.S. Pat. No. 6,261,548 as a factor capable of suppressing the proliferation of myeloid leukemia cells, such as murine M1 cells, and of promoting macrophage differentiation, suggesting its use as a non-proliferative therapeutic agent for suppressing certain forms of myeloid leukemia's and for modifying the function of macrophages involved in the response to infections. In the skin, LIF is a cytokine that is naturally produced by keratinocytes in vivo and in vitro (Paglia et al., Br. J. Dermatol., 134: 817-823, 1996), and it is known that this factor is in particular involved in the control of inflammatory processes associated with various skin pathologies, such as psoriasis (Bonifati et al., Arch. Dermatol. Res., 290: 9-13, 1998; Szepietowski et al., J. Dermatol, 28: 115-122, 2001), or certain allergies (Szepietowski et al., Contact Dermatitis., 36: 21-25, 1997).

However, to date, an effect of LIF, of a LIF analogue, of a LIF mimetic or of a product capable of stimulating the expression of endogenous LIF on the regenerative power of skin stem cells in culture has neither been described nor suggested, nor has their use in “anti-aging” or “anti-hair loss” cosmetic compositions or topical pharmaceutical compositions suited to promote skin cicatrization and/or regeneration, in particular for applications such as the treatment of burns.

The present invention therefore features the formulation, into cosmetic compositions, of an effective amount of a compound selected from among LIF, a LIF analogue, a LIF mimetic and a product capable of stimulating the expression of endogenous LIF, and mixtures thereof, as an active agent suited to maintain and/or stimulate the regenerative power of a population of human undifferentiated skin stem cells and/or epidermal progenitors.

In particular, the LIF, a LIF analogue, a LIF mimetic or a product capable of stimulating the expression of endogenous LIF, or mixtures thereof, is especially suited to:
(i) promote the capacity of the undifferentiated skin stem cells and/or epidermal progenitors for self-renewal and/or their capacity to proliferate;
(ii) and/or maintain and/or increase their capacity to generate a pluristratified epithelium, in particular a pluristratified epidermis, and/or to generate all or some of the skin appendages.

The term “skin appendages” in particular means the hair follicles, the sebaceous glands and the nails.

The LIF, a LIF analogue, a LIF mimetic or a product capable of stimulating the expression of endogenous LIF, or mixtures thereof, is also suited to:
(i) promote the capacity of the undifferentiated skin stem cells and/or epidermal progenitors for self-renewal and/or their capacity to proliferate;
(ii) and/or maintain and/or increase their capacity to generate a pluristratified epithelium, in particular a pluristratified epidermis, and/or to generate all or some of the skin appendages; in response to an exogenous stimulus and/or in response to a signal derived from the physiological environment of the cells in vivo.

The LIF, a LIF analogue, a LIF mimetic or a product capable of stimulating the expression of endogenous LIF, or mixtures thereof, contained in the compositions of the invention is in particular suited to:
stimulate the renewal of the epidermis and/or of all or some of the skin appendages; and promote in particular the radiance of the complexion;
prevent and/or combat cutaneous signs of aging, in particular prevent thinning of the skin and/or loss of firmness, of elasticity and/or of tonicity of the skin, and/or the formation of wrinkles and fine lines; and/or smooth out the microrelief of the skin in order to give it once again a young, smooth and firm appearance;
promote and/or increase the barrier function of the skin and improve especially the moisturization of the skin; in particular, prevent, improve and/or remedy conditions of dryness of the skin and/or of the mucous membranes, improve the aesthetic appearance and the comfort of the skin (suppleness), limit the formation of cracks or chapping on the lips, the hands, the face or the body;
revitalize the scalp and/or regenerate growth of the hair and/or of body hair.

This invention also features formulation of an effective amount of a compound selected from among LIF, a LIF analogue, a LIF mimetic and a product capable of stimulating expression of endogenous LIF, and mixtures thereof, into compositions suited for skin regeneration and/or cicatrization.

In particular, the subject compositions are suited for the treatment of burns, induced by excessive exposure to the sun (solar erythema) or subsequent to exposure to a strong source of heat (fire, hot water, etc.).

The present invention also features formulation of an effective amount of a compound selected from among LIF, a LIF analogue, a LIF mimetic and a product capable of stimulating the expression of endogenous LIF, and mixtures thereof, into compositions suited for the treatment of pathologies associated with altered desquamation, such as psoriasis and ichthyosis.

### DETAILED DESCRIPTION OF BEST MODE AND SPECIFIC/PREFERRED EMBODIMENTS OF THE INVENTION

The “LIF” in the compositions according to the invention may in particular be provided in the form of a purified LIF, of a recombinant LIF, of ground cellular material or a cell extract containing LIF, of a cell culture supernatant containing LIF or mixtures thereof.

A LIF of human origin will preferably be used.

Use may in particular be made of:
a LIF purified from organs, from tissues and/or from cells naturally expressing LIF [for example: pituitary gland cells (Ferrara et al., Proc. Natl. Acad. Sci., USA. 89: 698-702, 1992), dermal fibroblasts (Lorenzo et al., Clin. Immunol. Immunopathol., 70: 260-265, 1994), pulmonary fibroblasts (Elias et al., Am. J. Physiol., 266: L426435, 1994), placenta and endometrium (Kojima et al., Biol. Reprod., 50: 882-887, 1994), bone marrow stromal cells (Lorgeot et al., Cytokine., 9: 754-758, 1997), kidney cells (Morel et al., Cytokine., 12: 265-271, 2000), cardiomyocytes (Ancey et al. Cytokine., 18: 199-205, 2002)];
a recombinant LIF, as obtained in prokaryotic microorganisms (for example: bacterium, E. coli) or eukaryotic microorganisms (for example: yeast, Pichia pastoris, Saccharomyces) culture, possibly in the form of a fusion protein (for example: human recombinant LIF sold by Chemicon International Inc.);
a ground material or an extract of cells expressing LIF, in particular a ground material or an extract of nourishing cells (for example: murine fibroblasts of the 3T3 line) expressing LIF, or of cells genetically modified to express LIF, or else of cells stimulated to express LIF;
a cell culture supernatant containing LIF, such as a culture supernatant from 3T3 cells expressing LIF; or mixtures thereof.

As “product capable of stimulating the expression of endogenous LIF”, mention may be made, for example, of IL-1β, a cytokine described for its ability to induce the synthesis and secretion of LIF by fibroblasts, epithelial cells and smooth muscle cells of the human lung (Knight et al., Am. J. Respir. Cell Mol. Biol., 20: 934-841, 1999). Mention may also be made of TNF-α, and also the cyclic AMP analogue 8-bromoadenosine 3′: 5′ monophosphate (8BrcAMP), studied for their ability to induce the LIF promoter in a murine line of cells derived from the medullary stroma (Gollner et al., Cytokine., 11: 656-663, 1999).

According to an alternative, use may be made of any active principle capable, conversely, of repressing the activity of signaling pathways that are antagonists of the anti-differentiating effect of LIF, and in particular that involving the Stat5 protein. These are in particular antisense oligonucleotides, and/or methods of interference via small RNAs (siRNA), or else modulators of kinase and phosphatase activities involved in the activation of Stat molecules.

According to the invention, the term “LIF analogue” means in particular any modified LIF polypeptide or any LIF polypeptide fragment having LIF activity on skin stem cells, and/or epidermal progenitors, i.e., which is capable (i) of promoting the capacity of undifferentiated skin stem cells and/or epidermal progenitors for self-renewal and/or their capacity to proliferate, and (ii) of maintaining and/or increasing their capacity to generate a pluristratified epidermis.

The expression “modified LIF polypeptide having LIF activity” means in particular an LIF polypeptide having undergone one or more modifications, for example so as to increase its stability. The term “modification” is intended to mean any substitution, deletion and/or insertion of an amino acid or of a small number of amino acids, in particular by substitution of natural amino acids with non-natural amino acids or pseudo amino acids at positions such that the modifications do not significantly affect the biological activity of the LIF.

The modified LIF peptide may be obtained from a human LIF peptide sequence chosen from the Genbank AAA59217 sequence (195 aa), the GenBank AAA51699 sequence (202 aa), and the homologous sequences.

The term “homologous sequence” means a sequence that is identical to at least 70%, preferably at least 85%, and even more preferably at least 95%, of a defined peptide sequence, in the same species or in a different species; this is then referred to as an orthologous peptide sequence.

This modified LIF polypeptide may also be obtained from the sequences of the human LIF gene or cDNA (GenBank M63420, J05436, J03261, X13967), or from the sequences of the murine LIF gene or cDNA (GenBank M63419, J05435, X06381, X12810, S73374), according to conventional cloning and expression techniques.

The expression “LIF polypeptide fragment having LIF activity” means in particular a fragment of a sequence chosen from the Genbank AAA59217 sequence (195 aa), the GenBank AAA51699 sequence (202 aa) and the homologous sequences.

Said fragment will in particular be sufficiently large to reconstitute the tertiary structure of LIF exhibiting the LIF-R-binding and gp130-binding sites.

The polypeptide fragment may also be obtained, according to conventional cloning and expression techniques, from the sequences of the human LIF gene or cDNA (GenBank M63420, J05436, X13967, J03261), or from the sequences of the murine LIF gene or cDNA (GenBank X06381, M63419, J05435, X12810, S73374), and in particular from the coding sequences.

Such LIF analogues suitable for implementing the invention may thus be selected according to the method comprising the following steps:
a) a preparation of keratinocytes or of undifferentiated skin stem cells and/or epidermal progenitors is cultured (i) in the presence or (ii) in the absence of the test product;
b) the cells cultured according to (i) and (ii) are studied under a microscope;
c) the capacity of the product to promote the capacity of the skin stem cells and/or the epidermal progenitors for self-renewal and/or their capacity to proliferate is measured by comparing the number of cellular clones in the undifferentiated state obtained (i) in the presence or (ii) in the absence of the test product;
d) the product for which an increased number of cellular clones in the undifferentiated state is obtained in the presence of said product, compared with the number of cellular clones in the undifferentiated state in the absence of said product, is selected;
e) the capacity of said product to regenerate a pluristratified epidermis is then tested according to the following steps:
   a. a preparation of keratinocytes is seeded onto a dermal support (i) in the presence or (ii) in the absence of said test product;
   b. the structure of the epidermis reconstructed according to (i) and (ii) is observed under a microscope;
   c. the product for which an improved structure of the reconstructed epidermis is obtained in the presence of said product, compared with the structure of the epidermis in the absence of said product, is selected.

According to the invention, the term “LIF mimetic” means in particular any agonist of the LIF receptor (LIF-R) or alternatively any active agent, or cell extract or fraction of a cell extract capable of activating gp130 and/or the Jak/Stat and Ras/Map kinase signaling pathways, in particular the expression and/or the activity of Stat3.

Use may, for example, be made of antibodies that are LIF receptor agonists, synthetic peptides capable of interacting with LIF receptors and of activating them, or any active agent capable of inducing activation of the signaling pathways involved in the response to LIF, in particular the expression and/or the activity of Stat3, or the activation of Janus-associated tyrosine kinases (JAK).

In particular, use may be made of agents capable of inducing the formation of an LIFR-gp130 heterodimer, such as, for example, the cytokine oncostatin M (OSM), ciliary neurotrophic factor (CNTF) and cardiotrophin-1 (CT-1), which have strong tertiary structure homology with LIF.

Such LIF mimetics suitable for implementing the invention may be selected by means of conventional LIF-R-binding and gp130 activation assays.

These LIF analogues or LIF mimetics may be of natural or synthetic origin.

The term “natural origin” means a compound in the pure state or in solution at various concentrations, obtained by various extraction methods from a tissue (skin, etc.) of natural origin, in particular human epidermis, or from extracts of plant origin.

The term “synthetic origin” means a compound in the pure state or in solution at various concentrations, obtained chemically or by production in an organism after introduction into this organism of the elements required for this production.

It is in fact known that the cellular response to LIF involves a low-affinity receptor (glycoprotein of 190 kD, gp190) and a high-affinity receptor (glycoprotein of 130 kD, gp 130) (Taupin et al., J. Biol. Chem., 276: 47975-47981, 2001), and, downstream of these receptors, the Jak/Stat and Ras/Map kinase signaling pathways (Ernst et al., J. Biol. Chem., 274: 9729-9737, 1999; Burdon et al., Trends Cell Biol., 12: 432-438, 2002). The Stat3 signal transduction protein plays a predominant role in maintaining the undifferentiated state of murine ES cells in response to LIF (Niwa et al., Genes Dev., 12: 2048-2060, 1998; Matsuda et al., EMBO J., 18: 4261-4269, 1999), whereas the expression of the Stat5 protein is, conversely, associated with these cells entering into differentiation (Nemetz et al., Differentiation., 62: 213-220, 1998).

According to an alternative, use may also be made, in a composition or for preparing a composition according to the invention, of an LIF receptor (LIF-R) or a product capable of stimulating the expression of the endogenous LIF-R, preferably in combination with a LIF, a LIF analogue, a LIF mimetic or a product capable of stimulating the expression of LIF.

The compositions according to the invention may be in any of the pharmaceutical forms normally used in the cosmetics and dermatological fields, suitable for oral or topical administration, preferably for topical administration to the skin and/or the mucous membranes and/or the scalp.

The composition may be in a form suitable for the care or for the making-up of the skin and/or of the lips.

A formulation suitable for oral administration may be in the form of dragees, gelatin capsules, gels, emulsions, tablets, capsules or liquid solutions, in particular oral ampoules, for example. In particular, the active agent(s) according to the invention may be incorporated into any other forms of food supplements or enriched foods, for example food bars, or compacted or noncompacted powders.

Preferably, the effective amount of LIF, of a LIF analogue, of a LIF mimetic or of a product capable of stimulating the expression of endogenous LIF, or mixtures thereof, present in the composition ranges from 10⁻¹²% to 1% of the total weight of the composition, preferably from 10⁻⁹% to 0.1%, and even more preferably from 10⁻⁷% to 0.01% of the total weight of the composition.

If ground cellular material or a cell extract containing LIF is used, the effective amount of said ground material or of said extract present in the composition ranges from 10⁻⁹% to 1% of the total weight of the composition, preferably from 10⁻⁶% to 0.1%, and even more preferably from 10⁻⁴% to 0.01% of the total weight of the composition.

The invention also features compositions suitable for topical application to the skin, comprising, in a physiologically acceptable medium, at least an effective amount of a compound selected from among LIF, a LIF analogue, a LIF mimetic or a product capable of stimulating the expression of endogenous LIF, or mixtures thereof.

According to the invention, the term “physiologically acceptable medium” means a medium which is compatible with the skin and/or its integuments (eyelashes, nails, hair) and/or the mucous membranes (lips).

The compositions according to the invention are cosmetic compositions or pharmaceutical compositions, preferably cosmetic compositions.

The pharmaceutical compositions will preferably be dermatological compositions.

The effective amount of LIF, of a LIF analogue, of a LIF mimetic or of a product capable of stimulating the expression of endogenous LIF, or mixtures thereof, present in the composition preferably ranges from 10⁻¹²% to 1% of the total weight of the composition, preferably from 10⁻⁹% to 0.1%, and even more preferably from 10⁻⁷% to 0.01% of the total weight of the composition.

If ground cellular material or a cell extract containing LIF is used, the effective amount of said ground material or of said extract present in the composition ranges from 10⁻⁹% to 1% of the total weight of the composition, preferably from 10⁻⁶% to 0.1%, and even more preferably from 10⁻⁴% to 0.01% of the total weight of the composition.

The composition may in particular be in the form of an aqueous, optionally gelled, solution, of a dispersion of the lotion type, optionally two-phase lotion, of an emulsion obtained by dispersion of a fatty phase in an aqueous phase (O/W) or conversely (W/O), or of a triple emulsion (W/O/W or O/W/O) or of a vesicular dispersion of the ionic and/or nonionic type. These compositions are prepared according to the usual methods.

The LIF, a LIF analogue, a LIF mimetic or a product capable of stimulating the expression of endogenous LIF, or mixtures thereof, may also be formulated in compositions suitable for targeting to the deep layers of the epidermis, in particular for targeting to the basal layers of the epidermis or to the pilosebaceous unit. For example, the LIF may be (i) encapsulated in a coating such as microspheres, nanospheres, oleosomes or nanocapsules, or (ii) compartmentalized in a fatty phase containing the main constituents of sebum (squalene, triglycerides, aliphatic waxes, cholesterol waxes and free cholesterol), or structural constituents present in proportions similar to those present in sebum.

Particles called nanoparticles are in fact capable of crossing the superficial layers of the stratum corneum and/or of the follicular ostium, and of penetrating into the layers of the epidermis.

The advantage of a composition in which the fatty phase mimics the composition of sebum is to allow better availability of the active agent at the target organ, i.e., at the sebaceous gland.

The composition may have, for example, the appearance of a white or colored cream, of an ointment, of a milk, of a lotion, of a gel, of a serum, of a paste or of a foam, or may be in solid form (for example: stick) for application to the skin and/or the mucous membranes, such as the lips.

It may also be in the form of a lotion of the aqueous, aqueous-alcoholic or oily solution type, of an oil-in-water or water-in-oil or multiple emulsion, or of an aqueous or oily gel, or any other form suitable for application to the skin, the mucous membranes or the scalp.

In a known manner, the compositions according to the invention may also contain the adjuvants that are usual in the cosmetics or dermatological field, such as hydrophilic or lipophilic gelling agents, hydrophilic or lipophilic active agents, preservatives, antioxidants, solvents, fragrances, fillers, screening agents, pigments, odor absorbers and dyestuffs. The amounts of these various adjuvants are those conventionally used in the field under consideration, and are, for example, from 0.01 to 20% of the total weight of the composition. These adjuvants, depending on their nature, can be introduced into the fatty phase, into the aqueous phase or into the lipid vesicles. In any event, these adjuvants, and also the proportions thereof, will be chosen so as not to harm the desired properties of the LIF.

When the composition according to the invention is an emulsion, the proportion of the fatty phase can range from 5 to 80% by weight, and preferably from 5 to 50% by weight relative to the total weight of the composition. The oils, the emulsifiers and the coemulsifiers used in the composition in the form of an emulsion are chosen from those conventionally used in the field under consideration. The emulsifier and the coemulsifier are present, in the composition, in a proportion ranging from 0.3 to 30% by weight, and preferably from 0.5 to 20% by weight relative to the total weight of the composition.

As oils that can be used in the invention, mention may be made of hydrocarbons of mineral or synthetic origin (liquid petroleum jelly, isohexadecane), oils of plant origin (apricot kernel oil, liquid fraction of karite butter, avocado oil, soybean oil), oils of animal origin (lanolin), synthetic oils (perhydrosqualene, pentaerythrityl tetraoctanoate), silicone oils (cylcopentasiloxane and cyclohexasiloxane) and fluoro oils (perfluoro-polyethers). Use may also be made, as fats, of fatty alcohols (cetyl alcohol or stearyl alcohol), of fatty acids (stearic acid) and of waxes (carnauba wax, ozokerite, beeswax).

As emulsifiers and coemulsifiers that can be used in the invention, mention may be made, for example, of fatty acid esters of polyethylene glycol, such as PEG-100 stearate and PEG-20 stearate, and fatty acid esters of glycerol, such as glyceryl stearate.

As hydrophilic gelling agents, mention in particular may be made of carboxyvinyl polymers (carbomer), acrylic copolymers such as acrylate/alkyl acrylate copolymers, polyacrylamides, polysaccharides, natural gums and clays, and, as lipophilic gelling agents, mention may be made of modified clays such as bentones, metal salts of fatty acids, hydrophobic silica and polyethylenes.

As preservatives, mention may be made of esters of para-hydroxybenzoic acid, octane-1,2-diol, iodo-3-propynyl-2-butyl carbamate, phenoxyethanol and chlorhexidine gluconate.

As fillers, mention may be made, for example, of particles of polyamide (Nylon); microspheres of poly(methyl methacrylate); powders of ethylene-acrylate copolymer; expanded powders such as hollow microspheres, and in particular the microspheres made of a terpolymer of vinylidene chloride, of acrylonitrile and of methacrylate and sold under the name Expancel by the company Kemanord Plast; powders of natural organic materials such as starch powders, in particular powders of corn starch, of wheat starch or of rice starch, which may or may not be crosslinked, such as the powders of starch crosslinked with octenyl succinate anhydride; silicone resin microbeads such as those sold under the name Tospearl by the company Toshiba Silicone; silica; metal oxides such as titanium dioxide or zinc oxide; mica; and mixtures thereof.

As “solvents” mention may be made of hydrophilic organic solvents, lipophilic organic solvents, amphiphilic solvents or mixtures thereof.

Among the hydrophilic organic solvents, mention may be made, for example, of linear or branched lower monoalcohols having from 1 to 8 carbon atoms, such as ethanol, propanol, butanol, isopropanol or isobutanol, optionally oxyethylenated polyethylene glycols, polyols such as propylene glycol, isoprene glycol, butylene glycol, glycerol, sorbitol and its derivatives, glycol ethers and propylene glycol ethers.

As amphiphilic organic solvents, mention may be made of polyols such as propylene glycol derivatives.

As lipophilic organic solvents, mention may be made, for example, of fatty esters.

As “hydrophilic or lipophilic active agents” mention may be made of moisturizers, calmatives, depigmenting agents, anti-glycation agents, NO-synthase inhibitors, agents for stimulating the synthesis of dermal or epidermal macromolecules and/or for preventing their degradation, agents for stimulating fibroblast and/or keratinocyte proliferation or for stimulating keratinocyte differentiation, dermo-decontracting agents, tensioning agents, anti-pollution agents and/or free-radical scavengers, photoprotective agents, repairing and/or cicatrizing agents, antidandruff agents, and mixtures thereof.

Examples of such additional compounds are given below.

The term “moisturize” means:
either a compound that acts on the barrier function, with a view to maintaining the moisturization of the stratum corneum, or an occlusive compound. Mention may be made of ceramides, sphingoid-based compounds, lecithins, glycosphingolipids, phospholipids, cholesterol and its derivatives, phytosterols (stigmasterol, β-sitosterol, campesterol), essential fatty acids, 1,2-diacylglycerol, 4-chromanone, pentacyclic triterpenes such as ursolic acid, petroleum jelly and lanolin; C-glycosides;
or a compound that directly increases the water content of the stratum corneum, such as threalose and its derivatives, hyaluronic acid and its derivatives, glycerol, pentanediol, sodium pidolate, serine, xylitol, sodium lactate, poly(glyceryl acrylate), ectoin and its derivatives, chitosan, oligosaccharides and polysaccharides, cyclic carbonates, N-lauroylpyrrolidonecarboxylic acid, and N-α-benzoyl-L-arginine, urea and its derivatives;
or a compound that activates the sebaceous glands, such as DHEA, its 7-oxidized and/or 17-alkylated derivatives, sapogenins, and vitamin D and its derivatives;
or a compound that acts on the hydrolytic activities associated in particular with desquamation, such as Hepes and the sulfonic derivatives, jasmonic acid and its derivatives.

The depigmenting agents that can be incorporated into the composition according to the present invention comprise, for example, the following compounds: kojic acid; ellagic acid; arbutin and its derivatives, such as those described in EP-895,779 and EP-524,109; hydroquinone; aminophenol derivatives such as those described in WO 99/10318 and WO 99/32077, and in particular N-cholesteryloxycarbonyl-para-aminophenol and N-ethyloxycarbonyl-para-aminophenol; iminophenol derivatives, in particular those described in WO 99/22707; L-2-oxothiazolidine-4-carboxylic acid or procysteine, and its salts and esters; ascorbic acid and its derivatives, in particular ascorbyl glucoside; and plant extracts, in particular of liquorice, of mulberry and of skullcap, without this list being limiting.

The term “anti-glycation agent” means a compound that prevents and/or decreases the glycation of skin proteins, in particular of dermal proteins such as collagen. Examples of anti-glycation agents are extracts of plants of the family Ericaceae, such as an extract of blueberry (Vaccinium angustifolium); ergothioneine and its derivatives; and hydroxystilbenes and their derivatives, such as resveratrol and 3,3′,5,5′-tetrahydroxystilbene.

Examples of NO-synthase inhibitors that are suitable for use in the present invention comprise in particular an extract of the plant of the species Vitis vinifera that is in particular sold by the company Euromed under the name Leucocyanidines de raisins extra, or else by the company Indena under the name Leucoselect®, or, finally, by the company Hansen under the name Extrait de marc de raisin; an extract of a plant of the species Olea europaea that is preferably obtained from olive tree leaves and is in particular sold by the company Vinyals in dry extract form, or by the company Biologia & Technologia under the trade name Eurol BT; and an extract of a plant of the species Gingko biloba that is preferably a dry aqueous extract of this plant sold by the company Beaufor under the trade name Gingko biloba extrait standard.

Among the active agents that stimulate dermal macromolecules or prevent degradation thereof, mention may be made of those which act:
either on collagen synthesis, such as extracts of Centella asiatica; asiaticosides and derivatives; ascorbic acid or vitamin C and its derivatives; synthetic peptides such as iamin, the CL biopeptide or palmitoyloligopeptide sold by the company Sederma; peptides extracted from plants, such as the soybean hydrolysate sold by the company Coletica under the trade name Phytokine®; and plant hormones such as auxins and lignans;
or on elastin synthesis, such as the extract of Saccharomyces cerivisiae sold by the company LSN under the trade name Cytovitin®; and the Macrocystis pyrifera alga extract sold by the company Secma under the trade name Kelpadelie®;
or on glycosaminoglycan synthesis, such as the product of milk fermentation by lactobacillus vulgaris, sold by the company Brooks under the trade name Biomin yogourth®; the extract of brown alga Padina pavonica sold by the company Alban Müller under the trade name HSP3®; and the extract of Saccharomyces cerevisiae available in particular from the company Silab under the trade name Firmalift® or from the company LSN under the trade name Cytovitin®;
or on fibronectin synthesis, such as the extract of zooplankton Salina sold by the company Seporga under the trade name GP4G®; the yeast extract available in particular from the company Alban Müller under the trade name Drieline®; and the palmitoyl pentapeptide sold by the company Sederma under the trade name Matrixil®;
or on the inhibition of metalloproteinases (MMP) such as more particularly MMP 1, 2, 3 or 9. Mention may be made of: retinoids and derivatives, oligopeptides and lipopeptides, lipoamino acids, the malt extract sold by the company Coletica under the trade name Collalift®; extracts of blueberry or of rosemary; lycopene; isoflavones, their derivatives or plant extracts containing them, in particular extracts of soybean (sold, for example, by the company Ichimaru Pharcos under the trade name Flavosterone SB®), of red clover, of flax, of kakkon or of sage;
or on the inhibition of serine proteases such as leucocyte elastase or cathepsin G. Mention may be made of: the peptide extract of Leguminosa seeds (Pisum sativum) sold by the company LSN under the trade name Parelastyl®; heparinoids; and pseudodipeptides such as {2-[acetyl-(3-trifluoromethylphenyl)amino]-3-methylbutyrylamino}acetic acid.

Among the active agents that stimulate epidermal macromolecules, such as fillagrin and keratins, mention may in particular be made of the extract of lupin sold by the company Silab under the trade name Structurine®; the extract of Fagus sylvatica beech buds sold by the company Gattefosse under the trade name Gatuline®; and the extract of zooplankton Salina sold by the company Seporga under the trade name GP4G®.

The agents for stimulating fibroblast proliferation that can be formulated into the compositions according to the invention can, for example, be chosen from plant proteins or polypeptides, extracted in particular from soybean (for example an extract of soybean sold by the company LSN under the name Eleseryl SH-VEG 8® or sold by the company Silab under the trade name Raffermine®); and plant hormones such as giberrellins and cytokinins.

The agents for stimulating keratinocyte proliferation that can be included in the compositions according to the invention comprise in particular retinoids such as retinol and its esters, including retinyl palmitate; phloroglycinol; extracts of walnut cakes sold by the company Gattefosse; and extracts of Solanum tuberosum sold by the company Sederma.

The agents for stimulating keratinocyte differentiation comprise, for example, minerals such as calcium; the extract of lupin sold by the company Silab under the trade name Photopreventine®; sodium beta-sitosteryl sulfate sold by the company Seporga under the trade name Phytocohesine®; and the extract of corn sold by the company Solabia under the trade name Phytovityl®; and lignans such as secoisolariciresinol.

The compositions according to the invention can comprise dermo-decontracting agents, among which mention may in particular be made of alverine and its salts, in particular alverine citrate, sapogenins such as diosgenin and the natural extracts containing them (such as extracts of wild yam), certain secondary and tertiary carbonylamines, organic and inorganic salts of metals, in particular manganese gluconate, adenosine, and also the argireline R hexapeptide sold by the company Lipotec. Mention may also be made of extract of Boswellia seffata and certain fragrancing compositions with a dermo-decontracting effect.

The term “tensioning agent” means a compound capable of exerting tension on the skin, the effect of which is to temporarily fade out irregularities on the skin's surface, such as wrinkles and fine lines.

Among the tensioning agents that can be used in the composition according to the present invention, mention may in particular be made of:
(1) synthetic polymers, such as polyurethane latices or acrylic-silicone latices, in particular those described in EP-1-038,519, such as a propylthio(polymethyl acrylate), propylthio(polymethyl methacrylate) and propythio(polymethacrylic acid) grafted polydimethylsiloxane, or alternatively a propylthio(polyisobutyl methacrylate) and propylthio(polymethacrylic acid) grafted polydimethylsiloxane. Such grafted silicone polymers are sold in particular by the company 3M under the trade names VS 80, VS 70 or L021,
(2) polymers of natural origin, in particular (a) polyholosides, for example (i) in the form of starch derived in particular from rice, from corn, from potato, from cassaya, from pea, from Triticum aestivum wheat, from oat, etc. or (ii) in the form of carrageenans, alginates, agars, gellans, cellulose-based polymers and pectins, advantageously as an aqueous dispersion of gel microparticles, and (b) latices consisting of shellac resin, sandarac gum, dammar resins, elemi gums, copal resins, cellulose-based derivatives, and mixtures thereof,
(3) plant proteins and protein hydrolysates, in particular from corn, rye, Triticum aestivum wheat, buckwheat, sesame, spelt, pea, bean, lentil, soybean and lupin,
(4) mixed silicates, especially phyllosilicates and in particular laponites,
(5) wax microparticles chosen, for example, from carnauba wax, candelilla wax or alfalfa wax,
(6) colloidal particles of inorganic filler with a number-average diameter of between 0.1 and 100 nm, preferably between 3 and 30 nm, selected, for example, from among: silica, silica-alumina composites, cerium oxide, zirconium oxide, alumina, calcium carbonate, barium sulfate, calcium sulfate, zinc oxide and titanium dioxide.

The expression “anti-pollution agent” means any compound capable of trapping ozone, monocyclic or polycyclic aromatic compounds such as benzopyrene and/or heavy metals such as cobalt, mercury, cadmium and/or nickel. The term “free-radical scavenger” is intended to mean any compound capable of trapping free radicals.

As ozone-trapping agents that can be used in the composition according to the invention, mention may in particular be made of vitamin C and its derivatives, including ascorbyl glucoside; phenols and polyphenols, in particular tannins, ellagic acid and tannic acid; epigallocatechin and natural extracts containing it; extracts of olive tree leaf; extracts of tea, in particular of green tea; anthocyans; extracts of rosemary; phenol acids, in particular chorogenic acid; stilbenes, in particular resveratrol; sulfur-containing amino acid derivatives, in particular S-carboxymethylcysteine; ergothioneine; N-acetylcysteine; chelating agents such as N,N′-bis-(3,4,5-trimethoxybenzyl)ethylenediamine or one of its salts, metal complexes or esters; carotenoids such as crocetin; and various starting materials, for instance the mixture of arginine, histidine ribonucleate, mannitol, adenosine triphosphate, pyridoxine, phenylalanine, tyrosine and hydrolysed RNA, sold by Laboratoires Serobiologiques under the trade name CPP LS 2633-12F®, the water-soluble fraction of corn sold by the company Solabia under the trade name Phytovityl®, the mixture of extract of fumitory and of extract of lemon sold under the name Unicotrozon C49® by the company Induchem, and the mixture of extracts of ginseng, of apple, of peach, of wheat and of barley, sold by the company Provital under the trade name Pronalen Bioprotect®.

As agents for trapping monocyclic or polycyclic aromatic compounds that can be used in the composition according to the invention, mention may in particular be made of tannins such as ellagic acid; indole derivatives, in particular 3-indolecarbinol; extracts of tea, in particular of green tea, extracts of water hyacinth or Eichomia crassipes; and the water-soluble fraction of corn sold by the company Solabia under the trade name Phytovityl®.

Finally, as heavy metal-trapping agents that can be used in the composition according to the invention, mention may in particular be made of chelating agents such as EDTA, the pentasodium salt of ethylenediamine-tetramethylenephosphonic acid, and N,N′-bis(3,4,5-trimethoxybenzyl)ethylenediamine or one of its salts, metal complexes or esters; phytic acid; chitosan derivatives; extracts of tea, in particular of green tea; tannins such as ellagic acid; sulfur-containing amino acids such as cysteine; extracts of water hyacinth (Eichomia crassipes); and the water-soluble fraction of corn sold by the company Solabia under the trade name Phytovityl®.

The free-radical scavengers that can be used in the compositions according to the invention comprise, besides certain anti-pollution agents mentioned above, vitamin E and its derivatives, such as tocopheryl acetate; bioflavonoids; coenzyme Q10 or ubiquinone; certain enzymes such as catalase, superoxide dismutase, lactoperoxidase, glutathione peroxidase and quinone reductases; glutathione; benzylidinecamphor; benzylcyclanones; substituted naphthalenones; pidolates; phytantriol; gamma-orylzanol; lignans; and melatonin.

As indicated above, the compositions according to the invention can also contain UV-A and/or UV-B screening agents or photoprotective agents, in the form of organic or inorganic compounds, the latter being optionally coated so as to make them hydrophobic.

The organic photoprotective agents that are more particularly preferred are selected from among the following compounds: ethylhexyl salicylate, ethylhexyl methoxycinnamate, octocrylene, phenylbenzimidazole-sulfonic acid, benzophenone-3, benzophenone-4, benzophenone-5,4-methylbenzylidenecamphor, terephthalylidenedicamphorsulfonic acid, disodium phenyldibenzimidazoletetrasulfonate, 2,4,6-tris(diisobutyl 4′-aminobenzalmalonate)-s-triazine, anisotriazine, ethylhexyltriazone, diethylhexylbutamidotriazone, methylenebis(benzotriazolyl)-tetramethylbutylphenol, drometrizole trisiloxane, 1,1-dicarboxy-(2,2′-dimethylpropyl)-4,4-diphenylbutadiene, and mixtures thereof.

The inorganic photoprotective agents are selected from among pigments or even nanopigments (mean size of the primary particles: generally between 5 nm and 100 nm, preferably between 10 nm and 50 nm) of coated or uncoated metal oxides such as, for example, nanopigments of titanium oxide (amorphous or crystallized in rutile and/or anatase form), of iron oxide, of zinc oxide, of zirconium oxide or of cerium oxide, which are all UV photoprotective agents that are well known per se. Standard coating agents are, moreover, alumina and/or aluminum stearate. Such coated or uncoated metal oxide nanopigments are in particular described in EP-518,772 and EP-518,773.

The photoprotective agents are generally present in the compositions according to the invention in proportions ranging from 0.1 to 20% by weight relative to the total weight of the composition, and preferably ranging from 0.2 to 15% by weight relative to the total weight of the composition.

The repairing and/or cicatrizing agents can be selected from among vitamins A and derivatives, vitamin B3, provitamin B5, vitamin C and derivatives, vitamin E; the trace elements zinc, copper, manganese and magnesium; plant extracts from Centella asiatica, Mimosa tenuiflora, allantoin, Aloe vera; hyaluronic acid and alginates, and mixtures thereof.

The antidandruff agents can be selected from among antifungal and/or antibacterial agents. Mention may be made of: the zinc salt of pyridinethione or zinc omadine, 1-hydroxy-2-pyrrolidone derivatives, trihalocarbamides, triclosan, azole-containing compounds such as climbazole, ketoconazole, clotrinazole, econazole, isoconazole and miconazole, antifungal polymers such as amphotericin B or nystatin, and selenium sulfides.

The present invention also features a cosmetic regime or regimen for the treatment of dry and/or chapped and/or aged skin, characterized in that a cosmetic composition as defined above is administered or applied to the skin or the mucous membranes.

The application will preferably be located in the dry and/or chapped areas of the facial skin, the body skin or the skin of the lips, and in the areas where there are wrinkles on the face (forehead, crow's feet, nasolabial fold) and on the neck.

Also featured is a cosmetic regime or regimen for the treatment of hair loss, wherein a cosmetic composition as defined above is administered or applied to the scalp.

These compositions are suited to be used in the case of hair loss, whether this loss is of natural or drug-related origin, i.e., subsequent to the absorption of drugs for which hair loss is a side effect.

In order to further illustrate the present invention and the advantages thereof, the following specific examples are given, it being understood that same are intended only as illustrative and in nowise limitative. In said examples to follow, all parts and percentages are given by weight, unless otherwise indicated.

### BRIEF DESCRIPTION OF THE DRAWINGS

FIG. 1 shows an analysis of the effect of LIF on the clonogenic growth of stem cells and/or of epidermal progenitors. Cellular clones obtained from cells at passage 1 cultured for 8 days in the absence of exogenous LIF, and then fixed and stained (A); cellular clones obtained from keratinocytes at passage 1 cultured for 8 days in the presence of exogenous LIF at 1 ng/ml, and then fixed and stained (B); semi-quantitative analysis of the density of clones obtained under control conditions and in the presence of exogenous LIF (C).

FIG. 2 shows histological analyses of a reconstructed epidermis obtained in the presence or absence of ground material from 3T3 fibroblasts depleted or not depleted of LIF. Histological section of a reconstructed epidermis obtained in the presence of ground 3T3 material not depleted of LIF (positive control) (A); histological section of a reconstructed epidermis obtained in the absence of ground 3T3 material (negative control) (B); histological section of a reconstructed epidermis obtained in the presence of LIF-depleted ground 3T3 material (experimental condition) (C).

### EXAMPLES

### Example 1

### Undifferentiated Stem Cell and/or Epidermal Progenitor Growth Optimized in the Presence of LIF

Keratinocytes were isolated from an adult skin sample (breast plastic surgery).

After removal of the subcutaneous tissue using a scalpel, the skin sample is cut up into fragments of approximately 5 mm×5 mm, and then decontaminated by antibiotic treatment [Gentamycin (Life Technologies), 3 successive baths of 10 minutes in DMEM culture medium (Life Technologies)]. In order to allow separation of the dermis from the epidermis, the sample is then subjected to proteolytic treatment [dispase (Boehringer, Roche Diagnostics)+trypsin (Gibco, Invitrogen) overnight at 4° C.]. The epidermis is then separated from the dermis by dissection. The epidermal fragments separated from the dermal tissue are place in a solution of 0.05% trypsin-0.02% EDTA (Gibco, Invitrogen) (15 minutes at 37° C.). The preparation is stirred periodically in order to promote dissociation of the cells. The effect of the trypsin is then neutralized by adding a culture medium containing 10% serum (DMEM+10% serum). After neutralization of the trypsin (DMEM+10% serum), the cell preparation is homogenized mechanically (pipetting), and then filtered. The cell suspension is washed, and then resuspended in KGM medium (Clonetics). The cells in suspension are counted under a microscope using a Malassez cell. The viability of the samples is estimated by the Trypan blue (Life Technologies) exclusion method.

A step for enrichment in undifferentiated stem cells and/or epidermal progenitors was carried out by means of a step of rapid adhesion on a type I collagen substrate (selection of a population with a high capacity for adhesion, Adh⁺⁺⁺).

The undifferentiated stem cells and/or epidermal progenitors can in fact be separated from the more mature keratinocytes on the basis of their rapid adhesion property. This step allows the preparation to be pre-enriched with undifferentiated stem cells and/or epidermal progenitors.

The method of enrichment by adhesion on collagen is described in WO 03/038073 and scientific publication (Fortunel et al., J. Cell Sci., 116: 4043-4052, 2003).

The cell suspension is placed in culture flasks “coated” with type I collagen [the collagen is adsorbed onto the adhesion support by deposition of a liquid solution of collagen I (Sigma Chemical) diluted 2-fold in PBS, for at least 45 minutes, and then drying after removal of the surplus], at a density of 150 000 to 200 000 cells/cm². After 12 to 15 minutes, the keratinocytes that have not adhered are removed by washing in PBS buffer. The adherent cells, referred to as Adh⁺⁺⁺, thus selected are detached from the support by mild trypsinization (0.05% trypsin-0.02% EDTA (Gibco, Invitrogen) for 3 to 5 minutes at 37° C.). After neutralization of the trypsin (DMEM+10% serum), the cells are recovered, washed, and then resuspended in culture medium (in the present case, KGM medium). The fraction made up of the adherent cells, selected by this method, represents approximately 5 to 10% of the total keratinocytes of the epidermis.

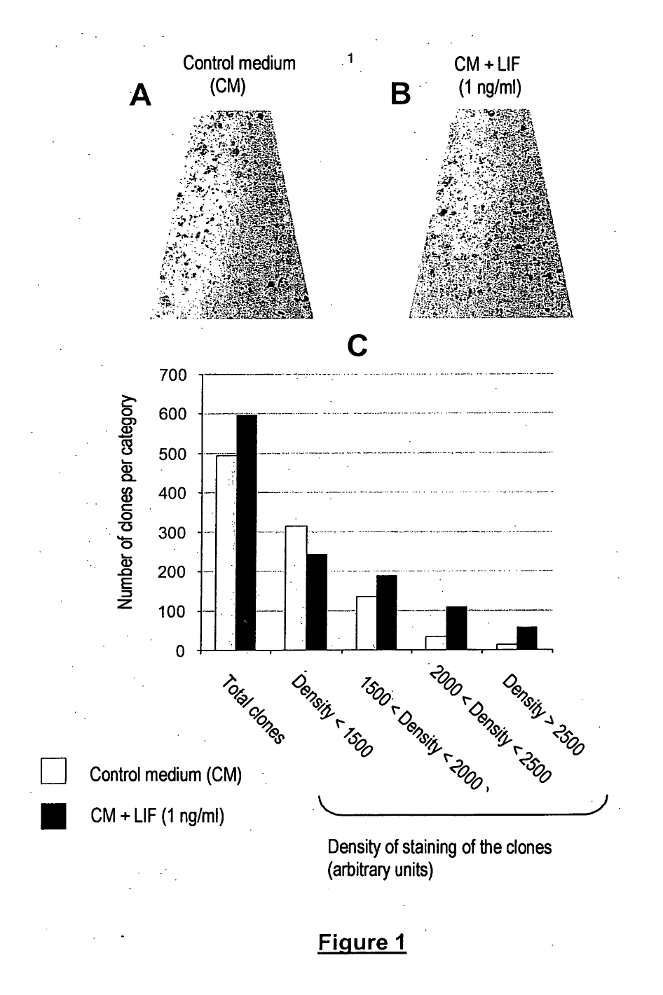

The cultures, initiated from Adh⁺⁺⁺ cells, were prepared in semi-defined culture medium (KGM Bullet Kit, Clonetics, Cambrex Bio Science Inc.), in the absence of feeder fibroblasts, and at low density (seedings at 2400 cells/cm²) in order to obtain quantifiable isolated cellular clones. At the first subculturing (passage 1), the cultures were divided into 2 batches: 1) condition identical to that described above; 2) addition of LIF at a concentration of 1 mg/ml (human recombinant LIF sold by Chemicon International Inc.). After culturing for 8 days, the cultures were fixed (70% ethanol) and stained (eosin and Giemsa) in order to analyze the characteristics of the cellular clones obtained under these 2 culture conditions (FIG. 1).

The comparative analysis of cultures performed in the absence or in the presence of LIF shows that this factor makes it possible to optimize the growth of undifferentiated stem cells and/or epidermal progenitors derived from a culture of Adh⁺⁺⁺ cells.

The macroscopic observation indicates an increase in the number of large densely-stained cellular clones (FIG. 1 A, B). The microscopic observation indicates that these clones whose development is promoted consists essentially of small cells exhibiting morphological characteristics associated with the undifferentiated state of stem cells and/or epidermal progenitors, which criterion reflects the fact that the cultures treated with LIF are “younger”. The increase in the number of large dense clones in response to LIF is confirmed by computerized image analysis, as represented in the histogram shown in FIG. 1C.

The use of LIF in a culture system therefore makes it possible to promote the multiplication of a population of human undifferentiated epidermal stem cells and/or epidermal progenitors.

### Example 2

### Positive Effect of LIF on Epidermal Reconstitution

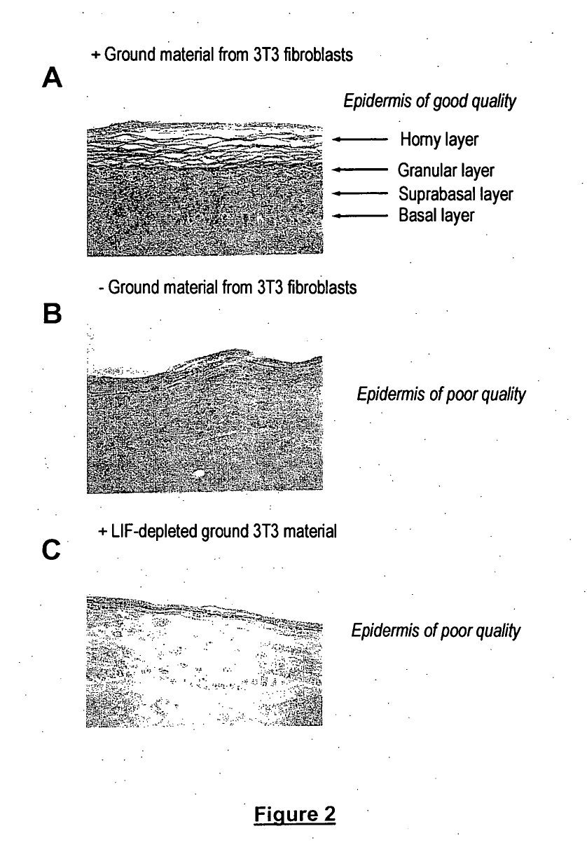

Ground material from 3T3 fibroblasts promotes epidermal reconstitution from stem cells and/or epidermal progenitors is cultured in vitro and stored in frozen form. The reconstructed epidermis is of good quality: it exhibits cellular organization and stratification similar to those of a natural epidermis (FIG. 2A).

In order to test the hypothesis that LIF is involved in this property, the LIF was depleted by immunoprecipitation and then the activity of the LIF-free ground material was compared with that of the non-depleted ground material.

The LIF-depletion of the ground 3T3 material is carried out using a polyclonal antibody produced in rabbits (anti-LIF Ab, Santa Cruz, ref. SC-20087). The ground 3T3 material is incubated with the anti-LIF Ab in excess at 4° C. for several hours in order to ensure correct binding of the LIF to the Ab. The free and LIF-bound Ab is then “trapped” by adding sepharose beads onto which proteins G have been adsorbed (proteins G have a high affinity for the Fc domains of Abs). Centrifugation makes it possible to separate the beads on which the LIF is retained from the LIF-depleted ground material.

The capacity of epidermal stem cells derived from a frozen library (isolated from a mammary skin sample) to generate a reconstructed epidermis was evaluated in the presence of ground material from 3T3 fibroblasts not depleted of LIF (positive control), in the absence of ground 3T3 material (negative control), and in the presence of LIF-depleted ground 3T3 material (experimental condition). The histological characteristics of the reconstructed epidermides obtained under each condition were compared on fixed and stained sections (FIG. 2).

Whereas the organotypic cultures supplemented with ground material from 3T3 fibroblasts (not depleted of LIF) make it possible to obtain reconstructed epidermides of good quality (FIG. 2A), i.e., a cellular organization and a stratification similar to those of a natural epidermis, the cultures prepared in the absence of ground material make it possible to obtain only epidermides that do not have the required histological characteristics (FIG. 2B).

The epidermides obtained in the absence of ground 3T3 material exhibit in particular the following anomalies:
substantial intercellular vacuoles and spaces;
poor stratification;
basal layer with small number of cells;
superbasal cells that are very stretched out;
incorrect orientation of the basal cells (parallel to the matrix);
little or no granular layer;
thin horny layer.

The cells of the experimental condition, cultured in the presence of LIF-depleted ground 3T3 material, behave in a similar manner to those cultured in the absence of ground material (FIG. 2C), which indicates that the depletion of LIF results in a loss of the beneficial properties of the ground material on the epidermal reconstruction.

Thus, the use of LIF, besides its capacity to promote the multiplication of said cells in an undifferentiated state, as shown in Example 1, makes it possible to maintain the organogenic potential of said cells, i.e., their capacity to generate a reconstructed epidermis of quality, exhibiting characteristics similar to those of natural epidermis.

### Example 3

### Compositions

Cosmetic Composition for Dry Skin:

| | |
|---|---|
| Recombinant LIF from Ex. 1 | 10⁻⁶% |
| 5-n-Octanoylsalicylic acid | 1% |
| Methylparaben | 0.1% |
| Propylparaben | 0.1% |
| Lanolin | 5% |
| Liquid petroleum jelly | 4% |
| Sesame oil | 4% |
| Cetyl alcohol | 5% |
| Glyceryl monostearate | 2% |
| Triethanolamine | 1% |
| Propylene glycol | 5% |
| Carbomer 940 | 0.1% |
| Water | qs |

Cosmetic Anti-Wrinkle Cream:

| | |
|---|---|
| Culture supernatant from 3T3 cells expressing LIF | 10⁻³% |
| Glyceryl stearate (emulsifier) | 2.00% |
| Polysorbate 60 (Tween 60 sold by ICI) (emulsifier) | 1.00% |
| Stearic acid | 1.40% |
| Triethanolamine (neutralizing agent) | 0.70% |
| Carbomer (Carbopol 940 sold by Goodrich) | 0.40% |
| Liquid fraction of karite butter | 12.00% |
| Perhydrosqualene | 12.00% |
| Preservative | 0.30% |
| Fragrance | 0.50% |
| Antioxidant | 0.05% |
| Water | qs 100% |

Care Stick for Chapped Lips:

| Culture supernatant from 3T3 cells expressing LIF | 10⁻³% |
|---|---|
| Lanolide | 13% |
| Microcrystalline wax | 20% |
| Ozokerite | 5% |
| Polar oil (castor oil) | 32% |
| Non-polar oil (polydecene) | 27% |
| Ascorbyl palmitate | 1% |
| Vitamin E | 1% |
| Fragrance | 1% |

Leave in Anti-Hair Loss Lotion:

| | | |
|---|---|---|
| Recombinant LIF from Ex. 1 | 1 | μg |
| Linoleic acid | 0.1 | g |
| Propylene glycol | 22.8 | g |
| 95° Ethanol | 55.1 | g |
| Purified | qs 100 | g |

Care Cream for Erythema:

| | |
|---|---|
| Recombinant LIF from Ex. 1 | 10⁻⁶% |
| Glyceryl stearate | 2.00% |
| Polysorbate 60 | 1.00% |
| Stearic acid | 1.40% |
| Glycyrrhetinic acid | 2.00% |
| Triethanolamine | 0.70% |
| Carbomer | 0.40% |
| Extract of Aloe vera | 2.00% |
| Sunflower oil | 10.00% |
| Antioxidant | 0.05% |
| Fragrance | 0.50% |
| Preservative | 0.30% |
| Water | qs 100% |

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A regime or regimen for maintaining and/or stimulating the regenerative power of a population of human undifferentiated skin stem cells and/or epidermal progenitors, comprising administering to an individual in need of such treatment, a thus effective amount of an active agent selected from the group consisting of leukemia inhibitory factor (“LIF”), an LIF analogue, an LIF mimetic, a product capable of stimulating the expression of endogenous LIF, and mixtures thereof, formulated into a physiologically acceptable medium thereof.

2. The regime or regimen as defined by claim 1, effective to (i) promote the capacity of the undifferentiated skin stem cells and/or epidermal progenitors for self-renewal and/or their capacity to proliferate and/or (ii) maintain and/or increase their capacity to generate a pluristratified epithelium and/or all or certain of the appendages of the skin.

3. The regime or regimen as defined by claim 1, effective to stimulate the renewal of the epidermal and/or all or certain of the appendages of the skin.

4. The regime or regimen as defined by claim 1, effective to prevent and/or combat the cutaneous signs of aging of the skin and/or loss of firmness, of elasticity and/or of tonicity of the skin and/or the formation of wrinkles and fine lines.

5. The regime or regimen as defined by claim 1, effective to promote the barrier function and/or moisturization of the skin.

6. The regime or regimen as defined by claim 1, effective to prevent and/or limit the formation of chapping on the lips, the hands, the face and/or the body.

7. The regime or regimen as defined by claim 1, effective to revitalize the scalp and/or regenerate growth of the hair and/or body hair.

8. The regime or regimen for promoting skin cicatrization and/or skin regeneration, comprising administering to an individual in need of such treatment, a thus effective amount of an active agent selected from the group consisting of leukemia inhibitory factor (“LIF”), an LIF analogue, an LIF mimetic, a product capable of stimulating the expression of endogenous LIF, and mixtures thereof, formulated into a physiologically acceptable medium thereof.

9. The regime or regimen as defined by claim 8, for treating skin burns.

10. A regime or regimen for treating a pathology associated with altered desquamation of the skin, comprising administering to an individual in need of such treatment, a thus effective amount of an active agent selected from the group consisting of leukemia inhibitory factor (“LIF”), an LIF analogue, an LIF mimetic, a product capable of stimulating the expression of endogenous LIF, and mixtures thereof, formulated into a physiologically acceptable medium thereof.

11. The regime or regimen as defined by claim 1, said active agent comprising an LIF of human origin, a purified LIF, a recombinant LIF, a ground cellular material or cell extract containing LIF, a cell culture supernatant containing LIF or mixture thereof.

12. The regime or regimen as defined by claim 1, said active agent comprising an amount of a modified LIF polypeptide or a LIF polypeptide fragment having LIF activity on the skin stem cells and/or the epidermal progenitors effective for promoting the capacity of the undifferentiated skin stem cells and/or epidermal progenitors for self-renewal and/or their capacity to proliferate and/or maintaining and/or increasing their capacity to generate a pluristratified epidermis.

13. The regime or regimen as defined by claim 1, said active agent comprising any agonist of the LIF receptor (LIF-R) or any active agent or cell extract capable of activating gp130 and/or the Jak/Stat and Ras/Map kinase signaling pathways

14. The regime or regimen as defined by claim 1, comprising topically applying said active agent/medium therefor onto the skin and/or mucous membranes, or onto the scalp.

15. The regime or regimen as defined by claim 1, comprising orally administering said active agent/medium therefor.

16. A cosmetic/dermatological composition suited for maintaining and/or stimulating the regenerative power of a population of human undifferentiated skin stem cells and/or epidermal progenitors, comprising a thus effective amount of an active agent selected from the group consisting of leukemia inhibitory factor (“LIF”), an LIF analogue, an LIF mimetic, a product capable of stimulating the expression of endogenous LIF, and mixtures thereof, formulated into a physiologically acceptable medium thereof.

17. The cosmetic/dermatological composition as defined by claim 16, said active agent comprising from 10⁻⁷% to 0.01% by weight thereof.

18. A topically applicable cosmetic/dermatological composition suited for maintaining and/or stimulating the regenerative power of a population of human undifferentiated skin stem cells and/or epidermal progenitors, comprising a thus effective amount of an active agent selected from the group consisting of leukemia inhibitory factor (“LIF”), an LIF analogue, an LIF mimetic, a product capable of stimulating the expression of endogenous LIF, and mixtures thereof, formulated into a topically applicable, physiologically acceptable medium thereof.

19. The cosmetic/dermatological composition as defined by claim 16, further comprising at least one hydrophilic or lipophilic active agent selected from the group consisting of moisturizers, calmatives, depigmenting agents, anti-glycation agents, NO-synthase inhibitors, agents for stimulating the synthesis of dermal or epidermal macromolecules and/or for preventing their degradation, agents for stimulating fibroblast and/or keratinocyte proliferation or for stimulating keratinocyte differentiation, dermo-decontracting agents, tensioning agents, anti-pollution agents and/or free-radical scavengers, photoprotective agents, repairing and/or cicatrizing agents, antidandruff agents, and mixtures thereof.
